(19) United States
(12) Patent Application Publication
(10) Pub. No.: US 2014/0275784 A1
(43) Pub. Date: Sep. 18, 2014

(54) CONSCIOUS SEDATION AIRWAY

(71) Applicant: Eileen T. Joyce, Village of Loch Lloyd, MO (US)

(72) Inventor: Eileen T. Joyce, Village of Loch Lloyd, MO (US)

(21) Appl. No.: 14/205,677

(22) Filed: Mar. 12, 2014

Related U.S. Application Data
(60) Provisional application No. 61/778,922, filed on Mar. 13, 2013.

(51) Int. Cl. A61M 16/04; A61M 16/00; A61B 1/00; A61M 16/06; A61B 17/02; A61B 13/00

(52) U.S. Cl. CPC A61M 16/0495; A61B 17/02; A61B 13/00; A61B 1/00154; A61M 16/0666; A61M 16/0078; A61M 16/0493
USPC 600/114; 600/239

(57) ABSTRACT

A conscious sedation airway, CSA, useable during conscious or unconscious sedation. The CSA includes an elongate body with an anterior flange depending therefrom. The body includes a central portion with superior and inferior plates extending a distance beyond a distal end of the body in an inferiorly curved arrangement. A midline opening and two lateral openings extend through the length of the central portion and are configured to receive apparatus, like endoscopes, cannula, and catheters. The superior and inferior plates aid to maintain a position of the tongue and to guide apparatus inserted through the midline opening toward the posterior oropharynx. The anterior flange prevents swallowing the CSA. The body does not extend into the pharynx to enable use in conscious patients and/or those with intact gag reflexes. And a nasopharyngeal airway can be placed without removing the CSA to allow bag ventilation of the patient.

### CROSS-REFERENCE TO RELATED APPLICATIONS

This application claims the benefit of U.S. Provisional Patent Application No. 61/778,922 filed Mar. 13, 2013, the disclosure of which is hereby incorporated herein, in its entirety, by reference.

### FIELD OF THE INVENTION

This invention relates to medical devices, and more particularly to a multi-functional conscience sedation airway that is also useable in the unconscious patient when desired.

### BACKGROUND

Oral or nasal airways are known in the art, and are used to maintain a patent airway in the unconscious patient. There are currently two popular airway devices that are used during airway management of the obstructed airway. The oropharyngeal airway, (OPA), is inserted into the mouth to prevent the tongue from resting against the posterior pharyngeal wall. The disadvantages to using the OPA include increased airway obstruction if placed incorrectly, trauma to soft tissue by catching the tongue or lips, and induced vomiting or coughing with a patient with intact airway reflex. The nasopharyngeal airway, (NPA), is also used to assist with the airway obstruction. It is less irritating; however, there are adverse effects as well. These effects include entry into the esophagus when the NPA is too long, injury to nasal mucosa, which can lead to bleeding and aspiration of blood clots and loss of the airway, e.g. swallowing of the airway, if there is no flange around the NPA.

Therefore, there is a need for a conscious sedation airway that can be inserted into the conscious patient during sedation cases and that can be used during gastrointestinal procedures. Such a conscious sedation airway that can also provide oxygen via a cannula, enable suction, enable insertion of a nasopharyngeal airway through the conscious sedation airway, and allow the patient to be safely bag ventilated if the patient becomes unconscious during sedation, (without necessitating the removal of the device from the patient) is also needed. The present invention accomplishes these objectives.

### SUMMARY

Embodiments of the invention are defined by the claims below, not this summary. A high-level overview of various aspects of the invention are provided here for that reason, to provide an overview of the disclosure, and to introduce a selection of concepts that are further described in the Detailed-Description section below. This summary is not intended to identify key features or essential features of the claimed subject matter, nor is it intended to be used as an aid in isolation to determine the scope of the claimed subject matter. In brief, this disclosure describes, among other things a conscious sedation airway (CSA) for insertion into the mouth.

The CSA has an elongate internal body that is insertable into a patient's mouth and includes an anterior flange depending from an anterior end thereof. The internal body is shortened compared to known devices and does not insert into the posterior oropharynx such that the device is less intrusive than the OPA or NPA. The internal body includes a central portion with superior and inferior plates extending a distance beyond a distal end of the body in an inferiorly curved arrangement. A midline opening and two lateral openings extend through the length of the central portion and are configured to receive apparatus, such as an endoscope, nasal cannula, catheters for suction, and a nasopharyngeal airway, among others. The inferior plate aids to maintain a position of the tongue and resists movement of the tongue distally toward the posterior oropharynx and thus causing obstruction thereof. The superior plate aids to guide apparatus inserted through the midline opening toward the posterior oropharynx.

The shortened length of the internal body enables the device to be inserted prior to sedation without triggering the patient's gag reflex. After sedation is initiated and if the patient becomes obstructed an NPA can be inserted through the midline opening to enable the patient to breath. The midline opening is dimensioned to enable insertion of the NPA therethrough but to obstruct passage of a proximal end of the NPA and prevent inhalation of the NPA into the patient's airway.

The CSA may also be employed during endoscopic examinations. The CSA can be inserted prior to sedation being initiated and secured with a head strap that couples to lateral apertures provided on the anterior flange of the CSA. Once the patient is adequately sedated the endoscope can be placed via the midline opening. The internal body of the CSA functions as bite block that prevents the patient from biting the endoscope inserted through the midline opening and potentially damaging the endoscope.

The configuration of the CSA also enables placement of oxygen cannula via one or both of the lateral openings, permits suctioning through the midline opening, and enables the provider to ventilate the patient with the CSA remaining in the mouth of the patient.

### DESCRIPTION OF THE DRAWINGS

Illustrative embodiments of the invention are described in detail below with reference to the attached drawing figures, and wherein:

FIG. 1 is a perspective view of a conscious sedation airway (CSA) illustrated with a nasal cannula and with straps secured thereto in accordance with an embodiment of the invention;

FIG. 2 is an elevational view of an anterior side of the CSA of FIG. 1 depicted without the nasal cannula and the strap;

FIG. 3 is an elevational view of a posterior side of the CSA of FIG. 1 depicted without the nasal cannula and the strap;

FIG. 4 is an elevational side view of the CSA of FIG. 1 depicted without the nasal cannula and the strap;

FIG. 5 is a diagrammatic side view of a patient with the CSA of FIG. 1 inserted into the mouth depicted in accordance with an embodiment of the invention;

FIG. 6 is a diagrammatic side view of a patient with the CSA of FIG. 1 inserted into the mouth, illustrated as used with a nasopharyngeal airway inserted through the CSA in accordance with an embodiment of the invention; and

FIG. 7 is a diagrammatic side view of a patient with the CSA of FIG. 1 inserted into the mouth, illustrated as used with an endoscope placed through the CSA in accordance with an embodiment of the invention.

### DETAILED DESCRIPTION

The subject matter of select embodiments of the invention is described with specificity herein to meet statutory requirements. But the description itself is not intended to necessarily limit the scope of claims. Rather, the claimed subject matter might be embodied in other ways to include different components, steps, or combinations thereof similar to the ones described in this document, in conjunction with other present or future technologies. Terms should not be interpreted as implying any particular order among or between various steps herein disclosed unless and except when the order of individual steps is explicitly described.

Referring initially to FIGS. 1-5, a conscious sedation airway (CSA) 10 is described in accordance with an embodiment of the invention. The CSA 10 is described herein with respect to use in humans, but such is not intended to restrict embodiments of the invention. In some embodiments, the CSA 10 can be configured for and employed in procedures in veterinary medicine. The construction and manufacturing of the CSA 10 is conducted using materials and methods known in the art and is not described in detail herein.

The CSA 10 includes an elongate internal body 12 with an anterior flange 14 disposed at or adjacent an anterior end thereof. The internal body 12 is configured for insertion in the mouth 15 of a patient 17 and includes a central portion 16, a superior plate 18, and an inferior plate 20. The central portion 16 includes a midline opening 22 and a pair of lateral openings 24, 26 that flank the midline opening 22. The openings 22, 24, 26 extend through the length of the central portion 16 and are open at each end thereof. Three openings 22, 24, 26 are described herein, however such is not intended to limit the scope of embodiments of the invention to a particular number or arrangement of openings. In embodiments, the provision of three openings 22, 24, 26 enables the provision of oxygen to the patient 17 as described below, as well as enables monitoring of carbon dioxide levels in the patient, and provides increased air exchange or ventilation of the patient's breathing.

The midline opening 22 has dimensions configured to receive apparatus such as a nasopharyngeal airway 46 (NPA)(FIG. 6), an endoscope 52 (FIG. 7), suction catheter, nasal cannula 30, or the like therein. The dimensions may also be configured to enable insertion of a portion of an apparatus through the midline opening 22 but to obstruct complete passage of the apparatus through the midline opening 22. For example, as depicted in FIG. 6, the midline opening 22 may be dimensioned to receive a tube portion 48 of an NPA 46 but to obstruct passage of a flared end 50 of the NPA. Such obstruction to complete passage of apparatus through the midline opening 22 resists or prevents inhalation or loss of the apparatus into the patient.

The lateral openings 24, 26 preferably have a diameter that is smaller than that of the midline opening 22, but the lateral openings 24, 26 may have any desired dimensions. As depicted in FIG. 1, the lateral openings 24, 26 may be dimensioned to receive a prong 28 of a nasal cannula 30, such as for provision of oxygen to a patient through the CSA. The lateral openings 24, 26 can be arranged such that one prong 28 is received by the lateral opening 24 and the second prong 28 is received by the midline opening 22 (as depicted in FIG. 1) or the lateral openings 24, 26 might be arranged to each receive a prong 28. The lateral openings 24, 26 may also be configured to receive a variety of other apparatus including catheters, cannula, and other equipment that might be employed during a procedure on a patient. The lateral openings 24, 26 may be dimensioned to receive the prong 28 of the nasal cannula 30 and/or any other apparatus in a friction-fit arrangement to assist retention of the prong 28 or other apparatus in the openings 24, 26. Or the prong 28 and/or other apparatus can be loosely or glidingly received therein.

The superior plate 18 and the inferior plate 20 extend along the superior or upper surface and the inferior or lower surface of the central portion 16 of the internal body 12, respectively. Both plates 18, 20 include a projection 32, 34 respectively that extends beyond a distal end of the central portion 16 a distance and curves or angles downwardly or inferiorly. The length of the plates 18, 20 is limited such that the plates 18, 20 do not extend into the posterior oropharynx 35. As such, the conscious patient 17 can tolerate the CSA without triggering the patient's gag reflex.

The plates 18, 20 may be coupled to the central portion 16 or may be integral therewith. In an embodiment, the plates 18, 20 are integrated into and form top and bottom surfaces of the central portion 16 and the superior and inferior projections 32, 34 extend from a distal end of the central portion 16. The plates 18, 20 in combination with the central portion 16 form a bite block that protects apparatus, such as an endoscope, inserted through one or more of the openings 22, 24, 26 from being bitten and/or damaged by the patient.

The downward curvature of the superior projection 32 on the superior plate 28 aids directing of apparatus inserted into one or more of the openings 22, 24, 26 toward the posterior oropharynx 35 as depicted in FIG. 7. For example, an endoscope 52 inserted through the midline opening 16 may contact the superior projection 32 and follow along a bottom surface thereof to be diverted toward the posterior oropharynx 35.

The inferior projection 34 assists maintaining the patient's tongue 37 in a forward anterior position. The inferior projection 34 curves downwardly into contact with the patient's tongue 37 to engage a surface thereof. The engagement with the tongue 37 resists the tongue 37 falling toward the posterior oropharynx 35 when the patient 17 is laid on their back and/or when the patient 17 is sedated and/or rendered unconscious.

The anterior flange 14 is configured to remain outside of the mouth 15 of the patient and to overlie the upper and lower lips of the patient to resist or prevent swallowing or inhalation of the CSA 10 by the patient. As depicted in FIG. 2, the anterior flange 14 includes a superior portion 36, an inferior portion 38, and a pair of lateral portions 40, but the anterior flange 14 may be configured as desired for a particular application without departing from the scope of embodiments of the invention described herein. The superior portion 36 extends a sufficient distance to at least partially overlie the patient's upper lip or jaw and the inferior portion 38 extends a sufficient distance to at least partially overlie the patient's lower lip or jaw. The lateral portions 40 include one or more apertures 42 configured to receive a strap 44. The strap 44 extends around the patient's head to maintain the CSA 10 in position in the patient's mouth 15 during sedation. The strap 44 may include elastic, rubber, or other resilient band, cord, strap, or other apparatus suitable for maintaining the position of the CSA 10.

With additional reference now to FIGS. 5-7, use of the CSA 10 in the patient 17 is described in accordance with an embodiment of the invention. As depicted in FIG. 5, the CSA 10 can be inserted into the mouth 15 of a patient 17 while the patient is conscious or after partial or full sedation. The superior and inferior portions 36, 38 of the anterior flange 14 rest on the lips of the patient 17. The anterior flange 14 thus prevents the patient 17 from swallowing the CSA 10. The internal body 12 rests between the upper and lower portion of the patients mouth 15. The internal body 12 can thus function as a bite block and to maintain a patent airway. The distance from the anterior flange 14 to the distal end of the internal body 12 is shorter than known airways used in the art and enables the conscious patient 17 to tolerate the CSA 10 without triggering the gag reflex. The inferior plate 20 and the inferior projection 34 thereof resists or prevents the tongue 37 from falling into the posterior oropharynx 35.

An NPA 46 can be inserted through the midline opening 22, for example, when the patient becomes obstructed and the provider needs another modality to maintain a patent airway, as depicted in FIG. 6. The NPA 46 extends beyond the internal body 12 into the posterior oropharynx 35. The midline opening 22 has a diameter smaller than that of the flange 50 of the NPA 46 to obstruct passage of the flange 50 through the midline opening 22. Swallowing and or aspiration of the NPA 46 are thus prevented.

As shown in FIG. 7, an endoscope 52 may be inserted through the midline opening 22 of the CSA 10. As described previously, the internal body 12 of the CSA 10 functions as a bite block to prevent the patient 17 from biting and/or damaging the endoscope 52 while inserted in the midline opening 22. During insertion of the endoscope 52 through the CSA 10, the superior projection 32 on the superior plate 18 may aid to guide the endoscope 52 toward the posterior oropharynx 35. As such, contact with the soft palate and other upper portions of the mouth 15 of the patient 17 that may cause harm or irradiation thereof may be reduced or avoided.

Many different arrangements of the various components depicted, as well as components not shown, are possible without departing from the scope of the claims below. Identification of structures as being configured to perform a particular function in this disclosure and in the claims below is intended to demarcate those structures as including a plurality of possible arrangements or designs within the scope of this disclosure and readily identifiable by one of skill in the art to perform the particular function in a similar way without specifically listing all such arrangements or designs. Embodiments of the technology have been described with the intent to be illustrative rather than restrictive. Alternative embodiments will become apparent to readers of this disclosure after and because of reading it. Alternative means of implementing the aforementioned can be completed without departing from the scope of the claims below. Certain features and subcombinations are of utility and may be employed without reference to other features and subcombinations and are contemplated within the scope of the claims.

What is claimed is:

1. A conscious sedation airway (CSA) apparatus for insertion into the mouth of a patient, the CSA comprising:
an internal body having a distal end that is insertable into the mouth of the patient;
an opening extending through the internal body and substantially parallel to the length of the internal body;
a superior projection extending from the distal end of the internal body and positioned superior to the opening; and
an inferior projection extending from the distal end of the internal body and positioned inferior to the opening, the inferior projection configured to contact the tongue of the patient to resist movement of the tongue toward the posterior oropharynx.

2. The CSA of claim 1, wherein one or both of the superior projection and the inferior projection turn downwardly toward the tongue of the patient when inserted into the mouth.

3. The CSA of claim 1, wherein the opening has dimensions suitable to receive one or more of an endoscope and a nasopharyngeal airway (NPA) therethrough.

4. The CSA of claim 1, further comprising:
one or more lateral openings extending through the internal body and aligned substantially parallel to the opening, the lateral openings having dimensions suitable to receive one or more of a cannula, a catheter, and a prong of a nasal cannula therein.

5. The CSA of claim 5, wherein at least one of the lateral openings is dimensioned to receive one or more of the cannula, catheter, and prong of the nasal cannula in a friction-fit manner.

6. The CSA of claim 1, further comprising:
an anterior flange that depends from an anterior end of the internal body, the anterior flange abutting the patient's lips when the internal body is inserted into the mouth of the patient and resists swallowing or inhalation of the CSA by the patient.

7. The CSA of claim 6, wherein the anterior flange includes a lateral aperture configured to receive a strap, the strap extendable around the patient's head to retain the internal body within the patient's mouth.

8. The CSA of claim 1, wherein the CSA does not extend into the posterior oropharynx when inserted into the mouth of the patient.

9. A conscious sedation airway (CSA) inserted into the mouth of a patient, the CSA comprising:
an internal body having a distal end that is disposed in the mouth;
an opening extending through the internal body and substantially parallel to the length of the internal body;
a superior projection extending from the distal end of the internal body and positioned superior to the opening;
an inferior projection extending from the distal end of the internal body and positioned inferior to the opening, the inferior projection contacting the tongue of the patient to resist movement of the tongue toward the oropharynx, the superior projection and the inferior projection terminating anterior to the oropharynx of the patient; and
an anterior flange depending from an anterior end of the internal body, the anterior flange abutting the patient's lips and resisting swallowing and inhalation of the CSA by the patient.

10. The CSA of claim 9, wherein the patient is conscious and the CSA does not trigger the patient's gag reflex.

11. The CSA of claim 9, wherein an endoscope is inserted through the opening and the CSA prevents biting of the endoscope by the patient.

12. The CSA of claim 9, wherein a tube portion of a nasopharyngeal airway is disposed in the opening and the opening is dimensioned to obstruct passage of a flared end of the nasopharyngeal airway through the opening.

13. The CSA of claim 9, wherein the patient is bag ventilated though the opening.

14. The CSA of claim 9, wherein the opening receives a prong of a nasal cannula in a friction-fit manner.

15. The CSA of claim 9, wherein the superior projection curves downwardly and guides an apparatus inserted through the opening toward the oropharynx.

16. A conscious sedation airway (CSA) for insertion into the mouth of a patient, the CSA comprising:
an internal body having a distal end that is disposable in the mouth;
a midline opening extending through the internal body and substantially parallel to a long axis of the internal body;
a secondary opening extending through the internal body substantially parallel to the midline opening, the secondary opening having a diameter that is smaller than the diameter of the midline opening;
a superior projection extending outwardly and downwardly from the distal end of the internal body and positioned superior to the opening;
an inferior projection extending outwardly and downwardly from the distal end of the internal body and positioned inferior to the opening, the inferior projection configured to contact the tongue of the patient to resist movement of the tongue toward the oropharynx, the superior projection and the inferior projection terminating anterior to the oropharynx of the patient when the internal body is disposed in the mouth; and
an anterior flange depending from an anterior end of the internal body, the anterior flange configured to abut the patient's lips and to resist swallowing and inhalation of the CSA by the patient.

17. The CSA of claim 16, wherein a first prong of a nasal cannula is inserted into the secondary opening and a second prong of the nasal cannula is inserted into the midline opening.

18. The CSA of claim 16, further comprising:
a tertiary opening extending through the internal body substantially parallel to the midline opening, the tertiary and the secondary openings disposed on opposing sides of the midline opening, and the tertiary opening having dimensions that are substantially similar to those of the secondary opening.

19. The CSA of claim 18, wherein the tertiary opening enables one or more of monitoring of carbon dioxide levels in the patient and increased air exchange through the internal body.

20. The CSA of claim 16, wherein the internal body includes a superior plate disposed along a superior side of the internal body and an inferior plate disposed along an inferior side of the internal body, and wherein the superior projection extends from the superior plate and the inferior projection extends form the inferior plate.
